(19) United States
(12) Patent Application Publication
(10) Pub. No.: US 2023/0364420 A1
(43) Pub. Date: Nov. 16, 2023

(54) A HEADSET FOR OCULAR DERMAL THERAPY

(71) Applicant: COSMOAESTHETICS PTY LTD, Kew (AU)

(72) Inventor: Masoud JAFARZADEH, Kew (AU)

(21) Appl. No.: 18/245,335

(22) PCT Filed: Sep. 15, 2021
(86) PCT No.: PCT/AU2021/051060

§ 371 Date: Mar. 14, 2023

(30) Foreign Application Priority Data
Sep. 17, 2020 (AU) 2020903326

(51) Int. Cl. A61N 1/32; A61N 1/04; A61N 5/06; A61H 23/02; A61F 7/00; A61H 7/00

(52) U.S. Cl. CPC A61N 1/328; A61N 1/0412; A61N 5/0616; A61H 23/02; A61F 7/007; A61H 7/004; A61N 2005/0652; A61N 2005/0663

(57) ABSTRACT

A headset for ocular dermal therapy is used for one or more of reducing dark eye circles, rejuvenating eyelid skin, boosting micro circulation, enhancing transdermal eye serum adsorption, reduction of allergy related skin conditions around the islands, improving skin tone, eye muscle relaxation and stress relief. The headset has a pair of periorbital contact eye pads which are controlled by a controller to administer ocular dermal therapy to the skin of the eyelids and surrounding the eye.

### FIELD OF THE INVENTION

This invention relates generally to dermal therapy, and, more particularly, a headset for ocular dermal therapy.

### BACKGROUND OF THE INVENTION

Dark circles under the lower eyelids are common in both men and women. Dark circles can enhance the appearance of ageing and are difficult to treat.

Dark circles may generally appear purple or blue to dark brown or black depending on skin colour and which may be caused by restricted blood vessels, hyperpigmentation, thinning of the skin around the eyes and the like.

There are various known causes for dark circles, including lack of sleep or poor sleeping habits, allergies (including hay fever), hyperpigmentation (which occurs when the body produces excess melanin), reduced fatty tissue levels around the eyes, thinning of the skin under the eyes, anaemia from iron deficiency, over exposure to sunlight, frequent rubbing of the eyes, age, smoking inherited genes, thyroid conditions, dehydration dermatitis and or the like.

Whereas these dark circles are rarely of cause for concern, reduction of the appearance thereof is desirous for cosmetic reasons.

The present invention seeks to provide a way to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein a headset for ocular dermal therapy which may be used for one or more of reducing dark eye circles, rejuvenating eyelid skin, boosting micro circulation, rehydration or treatment of dry eyes, enhancing transdermal eye serum adsorption, reduction of allergy related skin conditions around the islands, improving skin tone, eye muscle relaxation and stress relief.

The headset may comprise a housing held to the head by a headband. The headset may comprise a pair of periorbital contact eye pads. The pads are controlled by a controller to administer ocular dermal therapy to the skin of the eyelids and surrounding the eye.

The headset is worn such that the pads contact the eyes and surrounding tissue. Therapy controlled by the controller may last approximately 20 minutes and the headset may comprise sufficient rechargeable battery power supply for two hours of operation.

The pads may comprise flexible bodies having transverse plane curvature to fit and conform around the eyes.

Motion actuators may massage the pads over the eyes. The motion actuators may move or rotate the pads in opposite directions to minimise applied force to the housing. The left pad from the perspective of the wearer may rotate clockwise and the right pad may rotate oppositely anti-clockwise to push retained fluid away towards the infraorbital lymph nodes. The headset may comprise rotary to linear gearing which may be controlled by the controller to move the pads.

The pads may comprise electroporation contacts which apply an electric field to increase the permeability of skin cell membranes thereunderneath to enhance eye serum absorption applied either to the pads or directly to the skin.

The pads may comprise vibratory devices which may vibrate at frequencies in excess of 20 kHz to enhance microcirculation.

The pads may comprise thermal devices to apply heat up to but less than approximately 44° C. to enhance vasodilation and collagen stimulation.

The pads may comprise light sources to apply light including red light for boosting microcirculation and collagen remodelling, green light to even out skin tone and suppress melanin production, yellow light to lighten the skin and blue light to soothe and reduce skin sensitivity and inflammation.

The headset may conceal stereo speakers therein which may play music, including streamed from a connected Bluetooth device, during therapy.

The headset may comprise a hygienic and washable IPX6 housing and may be controllable by a touchscreen user interface.

The controller may store one or more therapy programs and/or user specific and/or user configurable settings which may control the timing and/or operation of at the electroporation contacts, thermal contacts, motion actuators, vibratory devices and/or light sources. Therapy programs may comprise various stages wherein, for example, a therapy program may commence with a first stage of boosting microcirculation wherein the controller controls the thermal devices to apply heat and the light source to apply red light and a further stage of reducing sensitivity wherein the controller controls the thermal devices not to apply heat and the light source to apply blue light.

The present headset applies ocular dermal therapy in a safe non-invasive pain-free manner.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a perspective view of a headset being worn for ocular dermal therapy;

FIG. 2 shows a further perspective view of the headset;

FIG. 3 shows a top plan view of the headset;

FIG. 4 shows a rear perspective view of the headset; and

FIG. 5 shows a functional control schematic of the headset in accordance an embodiment.

### DESCRIPTION OF EMBODIMENTS

A headset 100 comprises a pair of periorbital contact eye pads 102. The headset 100 further comprises a controller 101 to control the pads 102 to administer ocular dermal therapy.

The controller 101 may comprise a processor 103 for processing digital data. The controller 101 may further comprise a memory device 104 for storing digital information including digital data 105 and computer program code instructions. The computer program code instructions may be logically divided into a plurality of computer program code instruction controllers 106. In use, the processor 103 fetches these computer program code instructions and associated data 105 from the memory device 104 via a system bus 107 for interpretation and execution of the functionality described herein.

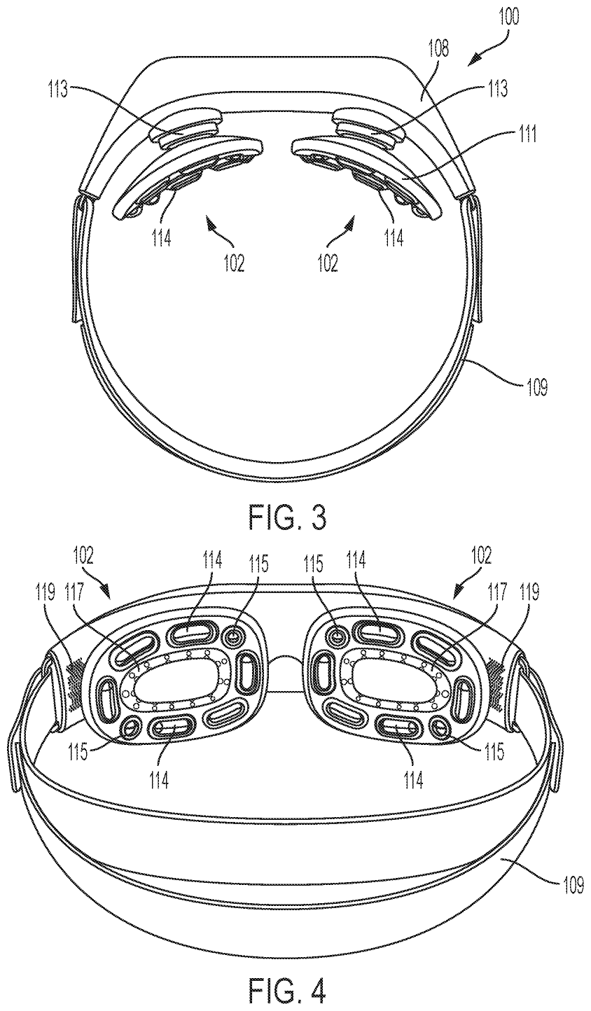

With reference to FIG. 3, the headset 100 may comprise a housing 108 and a rearward head attachment which may take the form of a length adjustable strap 109. The housing 108 may contain the controller 101 and a power source 110 which may take the form of a rechargeable battery. The pads 102 may extend rearwardly from the housing 108. The housing 108 may a gap between the pads 102 to allow for the bridge of the nose.

The pads 102 may comprise a flexible body 111 such as a silicon, rubber or the like to allow a rearward contact surface thereof to conform to the contours of the face of the wearer. Each pad 102 may be pivotally mounted to the housing 108 so as to be able to assume orientations conforming to the contours of the face of the wearer.

As is evident from FIG. 3, the body 111 may have a transverse plane curvature.

The rearward application surfaces of the pads 102 is sufficient to cover the eyes and the periorbital dermis. With reference to FIG. 4, the rearward application surface of each pad 102 may be generally laterally elongate.

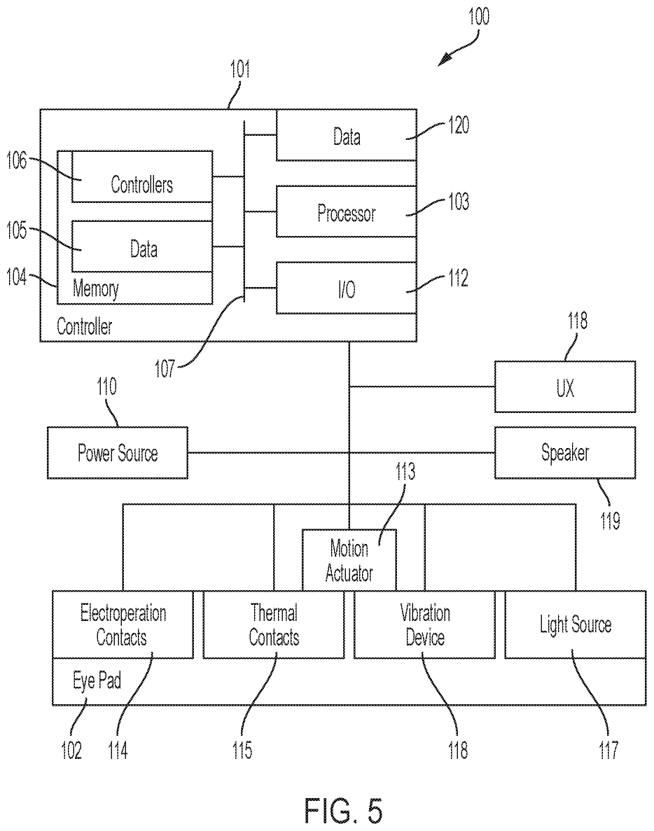

With reference to FIG. 5, the controller 101 may comprise an I/O interface 112 for interfacing with various peripherals.

The headset 100 may comprise a motion actuator 113 for each pad 102. Each motion actuator 113 is configured to move each respective pad 102 relative to the housing 108. The motion actuator 113 may comprise rotary to linear gearing driven by a DC motor to move the pad 102 linearly. Additionally, alternatively, the motion actuators 130 may rotate the pads 102.

In embodiments, the controller 101 may control the motion actuators 113 to move or rotate the pads 102 in opposite directions. For example, with reference to FIG. 4, the pads 102 may be moved by their respective motion actuators 113 in opposite directions left to right thereby massaging each eye whilst minimising resultant forces on the housing 108.

Additionally, or alternatively, the motion actuators 113 may rotate the pads 102 in opposite directions. With reference to the orientation of FIG. 4, the left pad 102 may rotate clockwise and the right pad may rotate oppositely anti-clockwise, to push retained fluid away towards the infraorbital lymph nodes.

The pads 102 may comprise exposed electroporation contacts 114 controlled by the controller 101 to apply an electric field to increase the permeability of the skin cell membranes thereunderneath. Eye serum may be applied to the pads 102 or to the eyes directly wherein the electroporation contacts 114 apply an electric field to increase the permeability of the skin cell membranes to more effectively absorb the eye serum.

As can be seen from FIG. 4, the electroporation contacts 114 may be generally concentrically arranged around the periphery of each pad 102.

The pads 102 may further comprise thermal devices to heat the skin thereunderneath. The thermal device may comprise a thermal contact 115. The thermal contact 115 may be heat conductive, such as being metallic, and may heat the skin by conduction. In alternative embodiments, the thermal contacts 115 may heat the skin by radiofrequency heating.

The thermal devices may be controlled by the controller 101 to apply heat up to but less than approximately 44° C. In this regard, each eye pad 102 may comprise a thermometer therein measuring heat via the contact surface area of the pad 102. As such, the controller 101 may apply heat via the thermal contacts depending on temperature measured by the thermometer. The thermal contacts 115 of each pad 102 may be generally concentrically arranged.

The pads 102 may further comprise a vibratory device 116 controlled by the controller 101. The vibratory device 116 may vibrate each pad 102 individually. In this regard, a vibratory device 116 may locate within the body of each pad 102. In embodiments, the vibratory device 116 comprises a micro motor turning an eccentric flywheel. In alternative embodiments, the vibratory device 116 is piezoelectric. The vibratory device 116 may operate at frequencies in excess of 20 Hz.

The pads 102 may further comprise a light source 117 controlled by the controller 101. The light source 117 may comprise different types of LEDs to apply light at different frequencies, including red, yellow, green and blue frequencies.

With reference to FIG. 4, the light source 117 may be a generally circular band of LEDs. The light source 117 may concentrically within the electroporation contacts 114 and the thermal contact 115.

The headset 100 may comprise speakers 119 for playing of music during therapy. With reference to FIG. 4, the housing 108 may comprise a pair of stereo speakers 119 behind grilles through a rear surface thereof. The controller 101 may comprise a data interface 120. In embodiments, the headset 100 may play music stream via the data interface 120 wherein, for example, the data interface 120 may be a Bluetooth data interface.

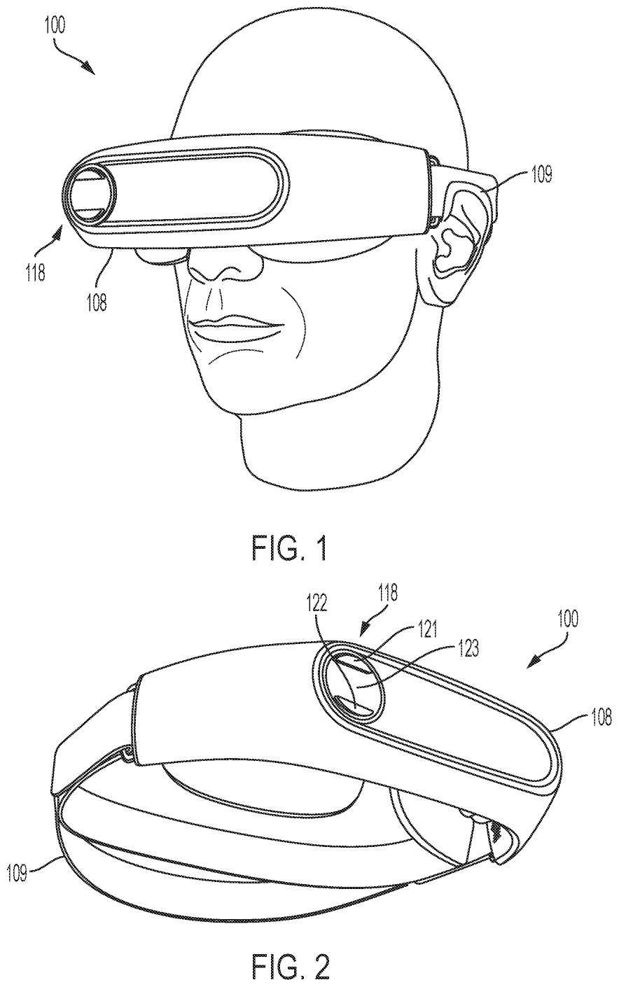

The headset 100 may comprise a user interface 118. With reference to FIGS. 1 and 2, the housing 108 may comprise the user interface 118 on a front surface thereof. User interface 118 may comprise physical and/or capacitive sensing buttons and/or digital displays. In the embodiments shown, the interface 118 comprises a music control button 121 and a power button 122. The interface 118 may further comprise a digital display 123 which may display the therapy program, therapy duration, therapy remaining time, operational settings and/or the like.

The data 105 may comprise one or more therapy programs and/or user specific and/or user configurable settings which may control the timing and/or operation of any one of the electroporation contacts 114, thermal contacts 115, motion actuators 113, vibratory devices 116 and light sources 117.

In embodiments, these therapy programs and/or settings may be selected and/or configured via a mobile phone device in operable communication with the controller 101 via the data interface 120.

In embodiments, the controller 101 may control the administration of a therapy program according to sensor feedback for example. For example, the controller 101 may monitor the thermometer to ensure a temperature setpoint has been reached prior to commencing the next stage of the therapy program.

The controller 101 may control the light source 117 depending on the type of therapy being administered. For example, therapy programs may be provided for applying light including red light for boosting microcirculation and collagen remodelling, green light to even out skin tone and suppress melanin production, yellow light to lighten the skin and blue light to soothe and reduce skin sensitivity and inflammation.

The controller 101 may control components of the pads 102 sequentially according to the various stages of the therapy program. For example, the controller 101 may control the light source 117 to apply different colours of light at various stages of a therapy program wherein, for example, a therapy program may commence with red light for boosting microcirculation and end with blue light for soothing and reducing skin sensitivity.

The controller 101 may control at least one of the light sources 117, electroporation contacts 114 and thermal contacts 115 synchronously. For example, when boosting microcirculation and collagen remodelling, the controller 101 may control the thermal contacts 115 to apply heat and red light whereas, when reducing skin sensitivity and inflammation, the controller 101 may control the thermal contacts 115 do not apply heat and to control the light sources 117 to apply blue light.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. A headset comprising a pair of periorbital contact eye pads and wherein the headset comprises a controller configured for controlling the operation of the pads to administer ocular dermal therapy.

2. The headset as claimed in claim 1, wherein the headset comprises a housing and rearward head attachment, wherein the pads extend rearwardly of the housing.

3. The headset as claimed in claim 1, wherein the pads comprise a flexible body.

4. The headset as claimed in claim 3, wherein each pad has transverse plane curvature.

5. The headset as claimed in claim 1, wherein a rearward contact surface of each pad is generally laterally elongate

6. The headset as claimed in claim 1, wherein each pad is pivotally mounted to the housing.

7. The headset as claimed in claim 1, wherein each pad comprises a respective motion actuator controlled by the controller.

8. The headset as claimed in claim 6, wherein the controller is configured to at least one of move and rotate the pads oppositely.

9. The headset as claimed in claim 8, wherein a left pad from a wearer perspective rotates clockwise and a right pad simultaneously rotates anti-clockwise.

10. The headset as claimed in claim 8, wherein the motion actuator comprises rotary to linear gearing.

11. The headset as claimed in claim 1, wherein the pads comprise electroporation contacts controllable by the controller to apply an electrical field.

12. The headset as claimed in claim 1, wherein the electric electroporation contacts occupy a periphery of each pad.

13. The headset as claimed in claim 1, wherein the pads comprise thermal devices controllable by the controller to apply heat.

14. The headset as claimed in claim 13, wherein thermal devices comprise conductive thermal contacts.

15. The headset as claimed in claim 13, wherein the thermal devices are configured to apply heat up to but less than approximately 44° C.

16. The headset as claimed in claim 15, wherein each pad comprises a thermometer therein measuring heat via a contact surface area thereof wherein the controller applies apply heat via the thermal contacts depending on temperature measured by the thermometer.

17. The headset as claimed in claim 13, wherein the thermal devices are radiofrequency heating devices.

18. The headset as claimed in claim 1, wherein the pads comprise a vibratory device controlled by the controller.

19. The headset as claimed in claim 18, wherein the vibratory devices operate at frequencies in excess of 20 Hz.

20. The headset as claimed in claim 1, wherein the pads comprise a light source controllable by the controller to apply light.

21. The headset as claimed in claim 20, wherein the light source is a generally circular band of LEDs.

22. The headset as claimed in claim 20, wherein the light source is controllable by the controller to apply different frequencies of light.

23. The headset as claimed in claim 22, wherein the different frequencies of light comprise at least one of red, green, yellow and blue light.

24. The headset as claimed in claim 20, wherein the light source is controllable by the controller to apply different frequencies of light at different time periods.

25. The headset as claimed in claim 1, wherein the housing comprises inbuilt stereo speakers.

26. The headset as claimed in claim 25, when the controller comprises a data interface and wherein the headset plays audio via the speakers received wirelessly via the data interface.

27. The headset as claimed in claim 1, wherein the controller comprises a memory device storing a therapy program and wherein the controller is configured for controlling the operation of the pads according to the therapy program.

28. The headset as claimed in claim 27, wherein the controller controls each pad according to stages of the therapy program.

29. The headset as claimed in claim 27, wherein the controller controls at least one of electroporation contacts, thermal devices and light sources of the pads according to stages of the therapy program.

30. The headset as claimed in claim 29, wherein the wherein, during a first stage of the therapy program, the controller controls the thermal devices to apply heat and the light source to apply red light whereas, at a further stage of the therapy program, the controller controls the thermal devices not to apply heat and the light source to apply blue light.

31. The headset as claimed in claim 1, wherein the headset comprises a user interface.

32. The headset as claimed in claim 31, wherein the headset comprises a frontward housing and wherein the user interface is on a front surface thereof.

33. The headset as claimed in claim 31, wherein the user interface comprises at least one of physical buttons, capacitive sensing buttons, and a digital display.

34. The headset as claimed in claim 31, wherein the user interface comprises a digital display which displays at least one of therapy program, therapy duration, therapy remaining time and operational settings.

35. A method of ocular dermal therapy using the headset as claimed in claim 1, the method comprising wearing the headset so that the pair of periorbital contact eye pads administer ocular dermal therapy.
